(19) United States
(12) Patent Application Publication
(10) Pub. No.: US 2022/0395605 A1
(43) Pub. Date: Dec. 15, 2022

(54) AIR SANITIZING MASKS

(71) Applicant: TEXAS INSTRUMENTS INCORPORATED, Dallas, TX (US)

(72) Inventor: Simon ZHAO, Dallas, TX (US)

(21) Appl. No.: 17/347,335

(22) Filed: Jun. 14, 2021

(51) Int. Cl. A61L 9/20; A61M 16/06; A61M 16/08

(52) U.S. Cl. CPC A61L 9/20; A61M 16/06; A61M 16/0875; A61L 2209/12; A61L 2209/14

(57) ABSTRACT

In some examples, a device comprises a sanitizing member including a hollow tube having a reflective inner surface and first and second ends. The sanitizing member includes an ultraviolet (UV) light-emitting diode (UV-LED) at the first end. The second end has an orifice exposing the reflective inner surface to an environment of the device. The device also includes a facemask having an air valve coupled to the sanitizing member.

### BACKGROUND

People wear various types of facial masks to mitigate the risk of pathogen exposure to themselves and to others. For example, surgeons wear masks during operations to mitigate the risk of infection in patients' open wounds. Scientists and researchers handling potentially dangerous biological agents wear masks to reduce the likelihood of infecting themselves. The general public may wear masks during viral outbreaks, such as during the cold and flu season or during pandemics.

### SUMMARY

In some examples, a device comprises a sanitizing member including a hollow tube having a reflective inner surface and first and second ends. The sanitizing member includes an ultraviolet (UV) light-emitting diode (UV-LED) at the first end. The second end has an orifice exposing the reflective inner surface to an environment of the device. The device also includes a facemask having an air valve coupled to the sanitizing member.

### BRIEF DESCRIPTION OF THE DRAWINGS

FIG. 1 is a perspective view of a mask device in various examples.

FIGS. 2A-2D are profile and perspective views of a hollow tube of a mask device in various examples.

FIGS. 3A, 3B and 3C are profile views of a hollow tube end cap of a mask device in various examples.

FIGS. 4A and 4B are profile views of an ultraviolet light-emitting diode (UV-LED) assembly in various examples.

FIG. 5 is a perspective view of a battery having a connector in various examples.

FIGS. 6A, 6B and 6C are profile views of a facemask of a mask device in various examples.

FIGS. 6D-6H are profile and profile cross-sectional views of air valves in a mask device in various examples.

FIG. 7 is a profile view of a mask device having multiple sanitizing members in various examples.

### DETAILED DESCRIPTION

Conventional masks are suitable for different purposes, and not all types of masks are effective in all circumstances. For example, some viruses are nanoscale viruses, and masks having pores that are several microns large may be ineffective in blocking the transmission of such nanoscale viruses. Besides efficacy, conventional masks may have other drawbacks, such as the collection of respiratory moisture, which promotes the growth of pathogens and raises the risk of illness. Some masks are effective in protecting the wearer, but not in protecting others in the vicinity of the wearer. Other masks do not adequately protect the wearer, but they provide a measure of protection for others in the vicinity of the wearer. Some masks are not reusable and other masks can be reused only a small number of times. Some masks restrict breathing to the point that the wearer is too uncomfortable to continue wearing the mask.

This description provides various examples of a mask device that resolves the foregoing challenges. The mask device includes a facemask having an elastic fastening member that is configured to fasten around a wearer's head, thus holding the facemask in place against the wearer's face. The facemask is formed of a non-porous material, such as rubber, metal, or plastic. The mask device also includes a sanitizing member that is adapted to be coupled to the facemask via a flexible hose. The sanitizing member may be a cylindrical tube with a reflective inner surface. The sanitizing member may include an ultraviolet (UV) light source (e.g., a UV-C light source) at a proximal end and an orifice at a distal end. The UV light source may be powered by a battery (e.g., a lithium-ion battery) and is configured to illuminate the length of the interior of the sanitizing member. Air enters the sanitizing member through the orifice and is sanitized as it flows along the length of the sanitizing member and toward the UV light source. By the time the air has reached the UV light source, the air has been substantially or fully sanitized. The sanitizing member includes another orifice adjacent to the UV light source through which the sanitized air enters the flexible hose and flows toward the facemask. The wearer then inhales the sanitized air. In this way, the wearer is protected against pathogens in the environment that may infect the wearer. The mask device may include a first valve (e.g., an exhaust valve) that opens as the wearer exhales. The mask device may include a second valve coupled to the flexible hose that closes as the wearer exhales to prevent carbon dioxide and/or unsanitary (e.g., exhaled) air from flowing into the flexible hose or the sanitizing member.

In some examples, the mask device also protects others in the wearer's vicinity from exposure to pathogens with which the wearer may be infected. Specifically, the mask device may include a second flexible hose coupled to the first valve, described above. The second flexible hose is coupled to a second sanitizing member. Responsive to the wearer exhaling, the exhaled air (which may be infectious) flows through the first valve (the second valve is closed), the second flexible hose, and to the second sanitizing member. As the exhaled air flows along the length of the second sanitizing member toward the orifice at the distal end of the second sanitizing member, the UV light in the second sanitizing member sanitizes the exhaled air. By the time the exhaled air reaches the orifice at the distal end of the mask device, the exhaled air has been sanitized. In this way, others in the wearer's environment are protected from possible infection.

The mask device described herein is superior to conventional masks for multiple reasons. First, the mask device does not rely on porous material to enable the wearer to breathe, and thus there is no risk that pathogens will penetrate the mask device and spread disease. Second, because a direct, unobstructed path exists between the wearer's respiratory system and the environment via the flexible hose and the sanitizing member, the wearer is unlikely to experience breathing difficulties or moisture collection. Third, because the sanitizing member sanitizes both inhaled and exhaled air, both the wearer and others in the wearer's environment are protected from pathogen exposure. Fourth, the mask device is reusable indefinitely. Examples of the mask device are now described with reference to the drawings.

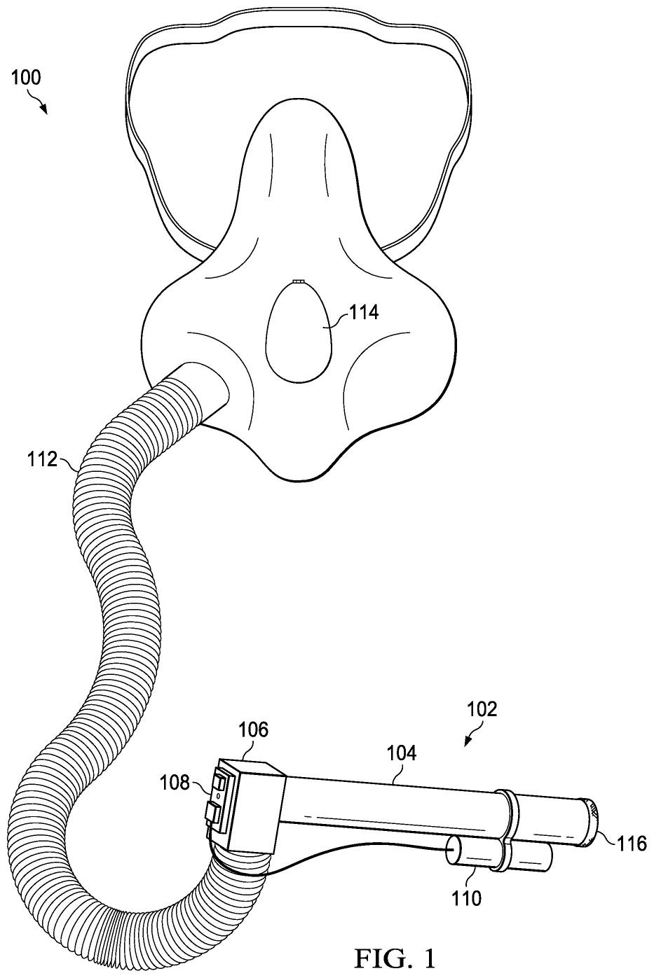

FIG. 1 is a perspective view of a mask device 100 in various examples. The mask device 100 includes a sanitizing member 102 having a hollow tube 104, a hollow tube end cap 106 coupled to a proximal end of the hollow tube 104, a UV-LED assembly 108 coupled to the hollow tube end cap 106, and a battery 110 coupled to the hollow tube 104 and the UV-LED assembly 108. The mask device 100 includes a flexible hose 112 that is coupled to the hollow tube end cap 106. The mask device 100 also includes a facemask 114 that is coupled to the flexible hose 112. An optional UV blocking filter 116, such as a UV blocking cloth, may cover a distal end of the hollow tube 104. Although not visible in the view of FIG. 1, the UV-LED assembly 108 includes a UV-LED that is powered by the battery 110 to emit UV-C light. The UV-LED is configured to direct the UV-C light from the UV-LED assembly 108, along the axis of the hollow tube 104, and toward the distal end of the hollow tube 104 where the optional UV blocking filter 116 may be located. Accordingly, the UV-LED of the UV-LED assembly 108 is configured to sanitize air in the hollow tube 104.

In examples, the hollow tube 104 has an inner surface that is reflective. For example, the inner surface of the hollow tube 104 includes aluminum. The inner surface of the hollow tube 104 may thus reflect the UV-C light emitted by the UV-LED and increase the sanitizing capabilities and efficiency of the mask device 100. In examples, the hollow tube 104 has a length that exceeds its maximum diameter. In examples, the hollow tube 104 has a length that is at least twice its maximum diameter. In examples, the hollow tube 104 has a length between two times and five times its maximum diameter. In examples, the hollow tube 104 has a length between five times and ten times its maximum diameter. In examples, the hollow tube 104 has a length between ten times and twenty times its maximum diameter. In examples, the hollow tube 104 has a length greater than twenty times its maximum diameter. In examples, the UV-LED assembly 108 may include multiple UV-LEDS to increase the sanitizing capabilities of the mask device 100.

In operation, a wearer wears the facemask 114. For example, the wearer wears the facemask 114 in a public area or other location with a heightened risk of pathogen transmission. Responsive to the wearer inhaling, air in the environment of the mask device 100 enters the distal end of the hollow tube 104. This air, which may contain pathogens, flows along the length of the hollow tube 104 toward the hollow tube end cap 106. As the air flows along the length of the hollow tube 104, the UV-C light emitted by the UV-LED sanitizes the air. The dimensions of the hollow tube 104, the power of the UV-LED, the number of UV-LEDS, and other variables may be determined so the air is sanitized by the time the air reaches the hollow tube end cap 106. Examples of such determinations are described below. The flexible hose 112 provides the sanitized air from the hollow tube end cap 106 to the facemask 114. The wearer may inhale the sanitized air.

In examples, and as described below, the facemask 114 includes an air valve that prevents exhaled air from flowing through the flexible hose 112 and toward the sanitizing member 102. In examples, and as described below, the facemask 114 includes an air valve through which exhaled air is expelled to an environment of the mask device 100. In examples, and as described below, the facemask 114 includes an air valve that is coupled to another flexible hose, and this flexible hose is coupled to another sanitizing member. This sanitizing member is configured to use UV-C light to sanitize air exhaled by the wearer, thereby preventing the potential transmission of pathogens from the wearer to others in the vicinity of the wearer.

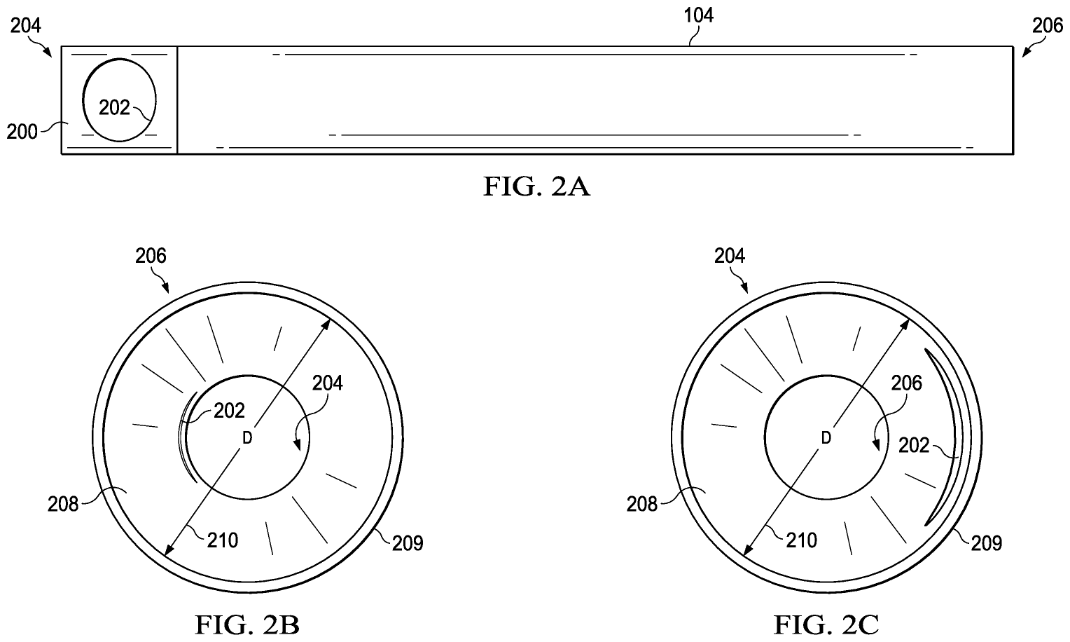

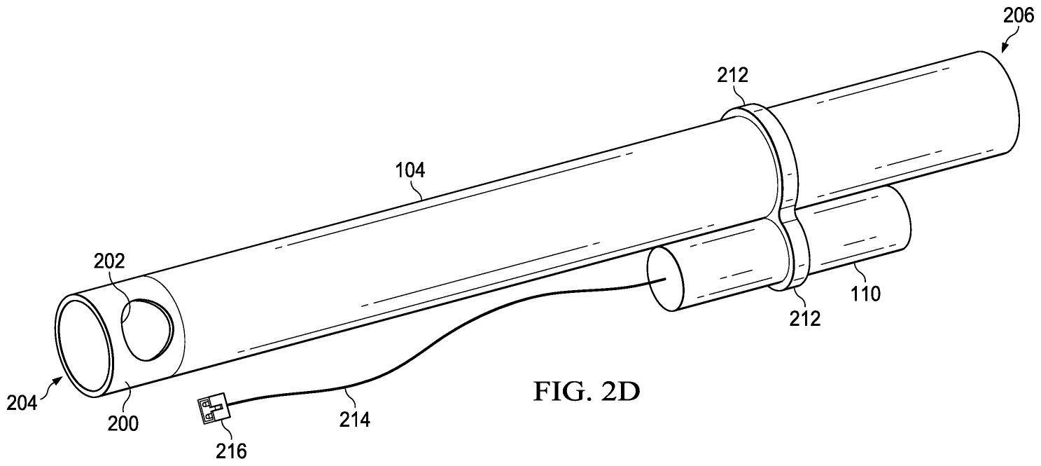

FIG. 2A is a profile view of the hollow tube 104 in various examples. In some examples, the hollow tube 104 has a cylindrical cross-section. In some examples, the hollow tube 104 has a polygonal cross-section (e.g., a rectangle, a square, or any other suitable shape). The hollow tube 104 may include an insertion member 200 adapted to be coupled to the hollow tube end cap 106, and the hollow tube 104 also includes an orifice 202 in the insertion member 200. In examples, the orifice 202 has a total area that is less than or approximately equal to the cross-sectional area of the interior of the hollow tube 104. The hollow tube 104 may be composed of any suitable material, including plastics, metals, etc. In some examples, an interior surface of the hollow tube 104 is reflective (e.g., aluminum). The hollow tube 104 includes a proximal end 204 and a distal end 206. In some examples, the proximal end 204 and distal end 206 include orifices, each of which may have an area approximately equal to the cross-sectional area of the interior of the hollow tube 104.

FIG. 2B is a profile view of the hollow tube 104 in various examples. The view of FIG. 2B is through the orifice of the distal end 206 and toward the proximal end 204. As described, an interior surface 208 of the hollow tube 104 is reflective and may be composed of aluminum. The hollow tube 104 has a wall 209 and an inner diameter 210. The inner diameter 210 is useful to calculate a cross-sectional area of the hollow tube 104, which may be useful in determining pathogen-elimination efficacy, as described below. FIG. 2C is a profile view of the hollow tube 104 in various examples. The view of FIG. 2C is through the orifice of the proximal end 204 and toward the distal end 206.

FIG. 2D is a perspective view of the hollow tube 104 and the battery 110 in various examples. In some examples, the battery 110 is a lithium ion battery, although other types of batteries are contemplated and included in the scope of this description. A clamp 212 is adapted to be coupled to the battery 110 and to the hollow tube 104. A battery wire 214 extends from the battery 110 and terminates at a connector 216. The battery wire 214 and the connector 216 are configured to provide power from the battery 110 to the UV-LED assembly 108 (FIG. 1).

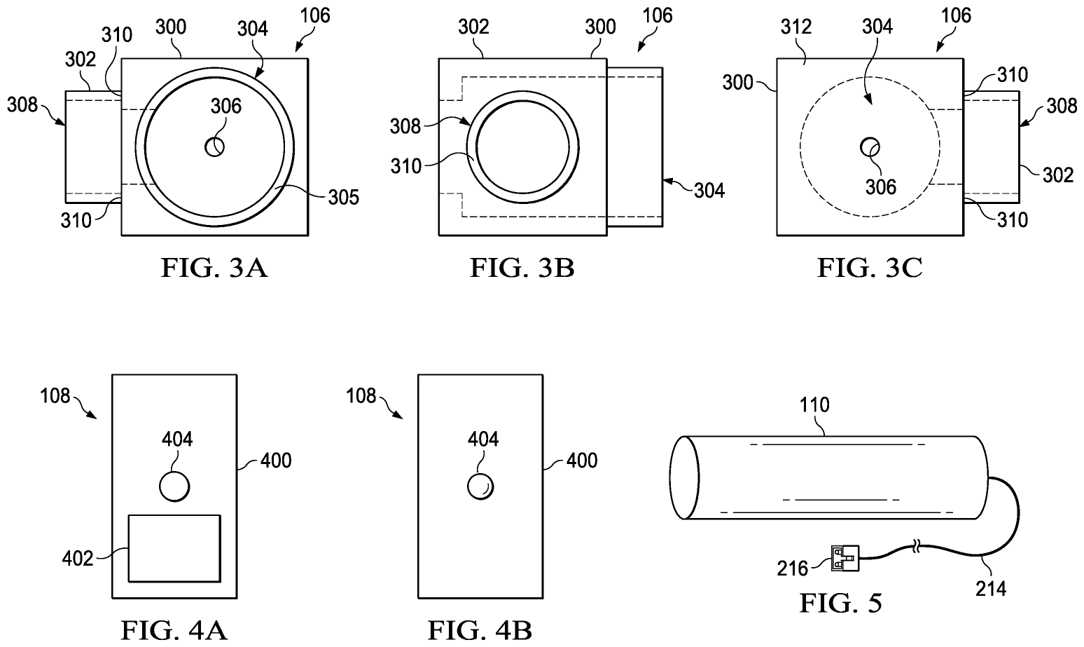

FIG. 3A is a profile view of the hollow tube end cap 106 in various examples. The hollow tube end cap 106 may include a member 300 and a member 302 coupled to the member 300. Each of the members 300, 302 may be a rectangular prism, although other shapes are contemplated. The member 300 includes a port 304, and the member 302 includes a port 308. The port 304 is adapted to accept the insertion member 200 of the hollow tube 104. The member 300 includes a shoulder 305 in the port 304 to preclude over-insertion of the insertion member 200. The insertion member 200 is radially oriented so the orifice 202 aligns with the port 308. The member 300 includes an orifice 306 through which the UV-LED described herein may illuminate the interior of the hollow tube 104. For example, the UV-LED assembly 108 may be coupled to the member 300 so the UV-LED on the UV-LED assembly 108 is aligned with the orifice 306. In examples, the diameter of the orifice 306 is smaller than the diameter of the hollow tube 104.

FIG. 3B is a profile view of the hollow tube end cap 106 in various examples. The member 302 includes the port 308 and a shoulder 310 in the port 308. The port 304 and the port 308 are in fluid communication with each other, meaning that a gas, such as air, may flow freely between the ports 304 and 308. The port 308 is adapted to accept the flexible hose 112, and the shoulder 310 prevents over-insertion of the flexible hose 112. As a result of the insertion member 200 being coupled to the port 304 and the flexible hose 112 being coupled to the port 308, fluid communication is established between the hollow tube 104 and the flexible hose 112 through the hollow tube end cap 106. In some examples, the insertion member 200, flexible hose 112, and the ports 304 and 308 may include features that facilitate coupling, such as threads, latches, etc. FIG. 3C is a profile view of the hollow tube end cap 106 in various examples. The UV-LED assembly 108 may be coupled to a surface 312 of the member 300 so the UV-LED on the UV-LED assembly 108 is aligned with the orifice 306.

FIG. 4A is a profile view of the UV-LED assembly 108 in various examples. The UV-LED assembly 108 includes a circuit board 400, a UV-LED driver 402 coupled to the circuit board 400, and a UV-LED 404 coupled to the circuit board 400. The UV-LED driver 402 is coupled to the UV-LED 404 via the circuit board 400 and provides adequate current (e.g., 350 milli-Amps) to the UV-LED 404 via the circuit board 400. In some examples, the UV-LED 404 provides UV-C light. The view of FIG. 4A shows the UV-LED 404 but does not show the UV-LED 404 bulb. FIG. 4B is a profile view of the UV-LED assembly 108 in various examples. FIG. 4B shows the UV-LED 404 bulb. In some examples, the UV-LED 404 bulb aligns with and/or fits inside the orifice 306 (FIGS. 3A and 3C). The circuit board 400 includes multiple pins that are coupled (e.g., soldered) to pins of the UV-LED driver 402, the UV-LED 404, and the connector 216 (described below with reference to FIG. 5). The UV-LED assembly 108 may be coupled to the surface of the member 300 shown in FIG. 3C using any suitable technique, such as an adhesive, screws, a fastener, etc. In some examples, the UV-LED assembly 108 may include multiple UV-LEDs, and in such examples, the surface 312 in FIG. 3C may include multiple orifices 306 to accommodate the multiple UV-LEDs.

FIG. 5 is a perspective view of the battery 110 in various examples. As described above, the battery 110 may be a lithium-ion battery and may provide any suitable output power that is adequate to power the UV-LED 404 for a target or threshold length of time. The capacity of the battery 110 may vary based on the number of UV-LEDs being powered, the wattage of the UV-LEDs, the target or threshold length of time for which the UV-LED(S) are to be illuminated, etc. The battery 110 includes a battery wire 214 that is coupled to the connector 216. As described above, the battery 110 may be coupled to the hollow tube 104 using the clamp 212 (FIG. 2D), and the connector 216 may be coupled to the UV-LED assembly 108 (e.g., to a same surface of the UV-LED assembly 108 as the UV-LED driver 402).

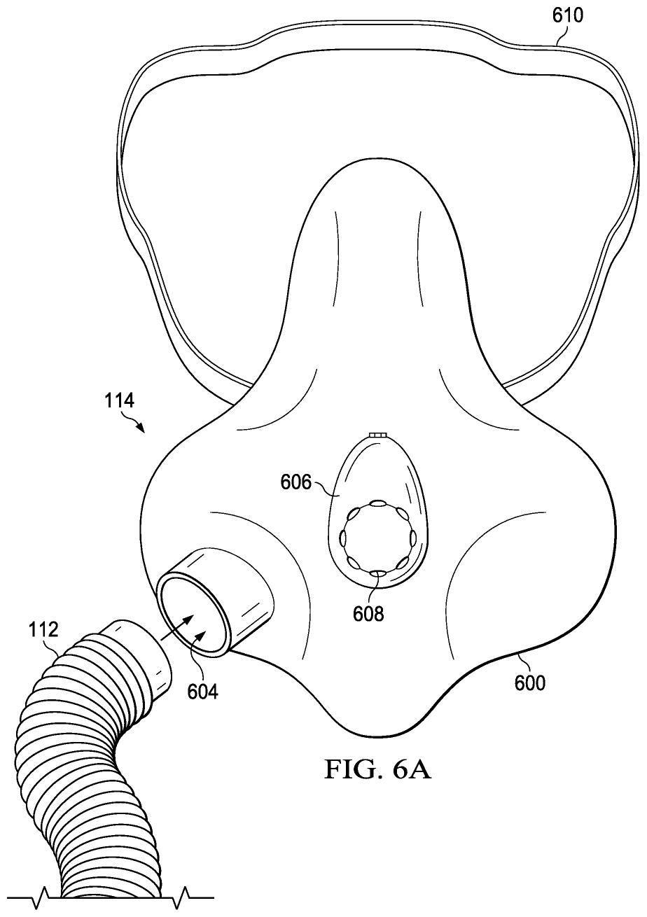

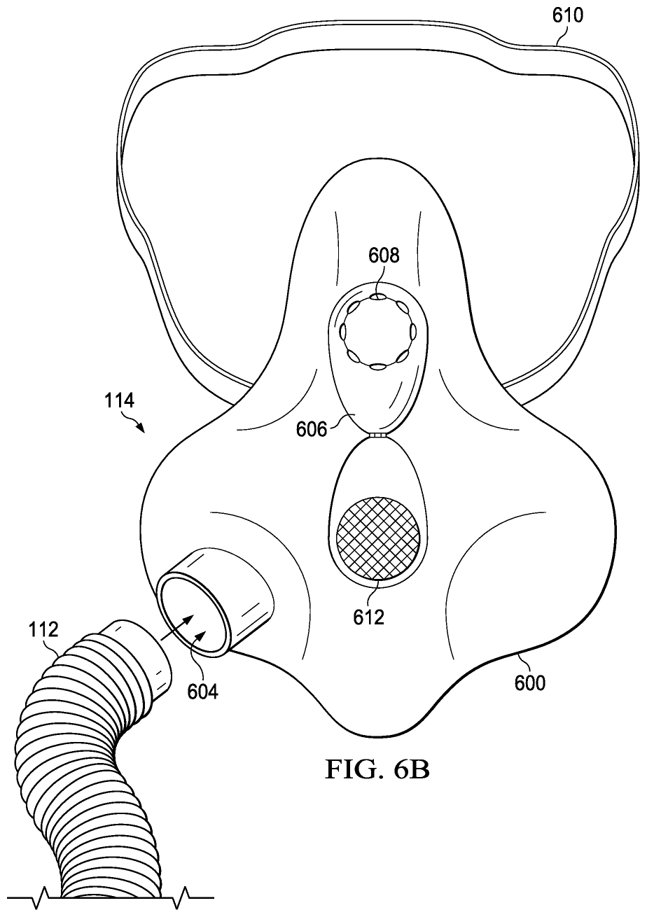

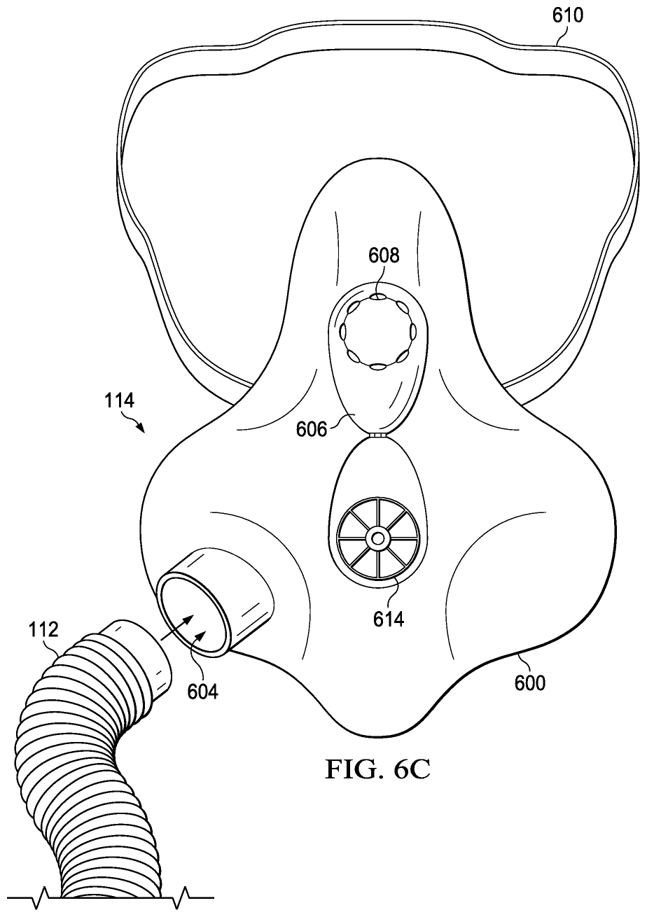

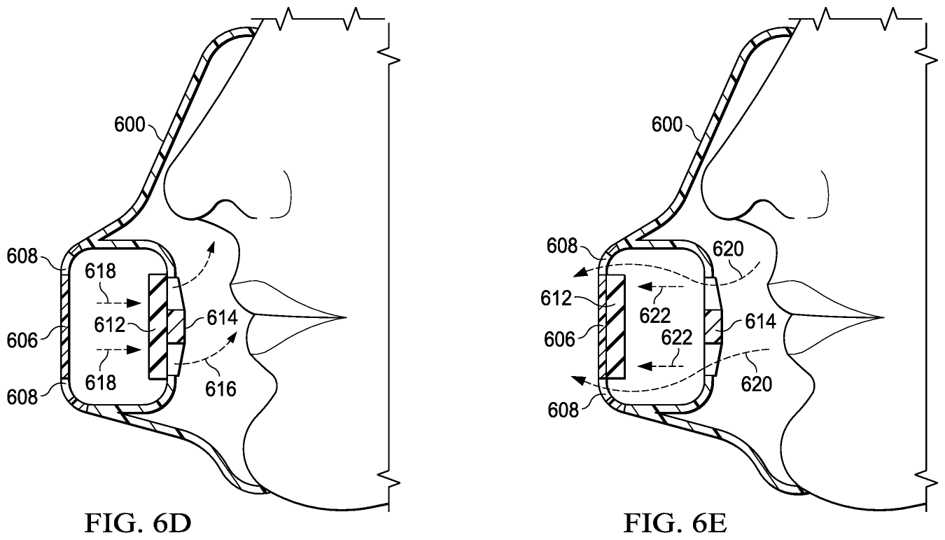

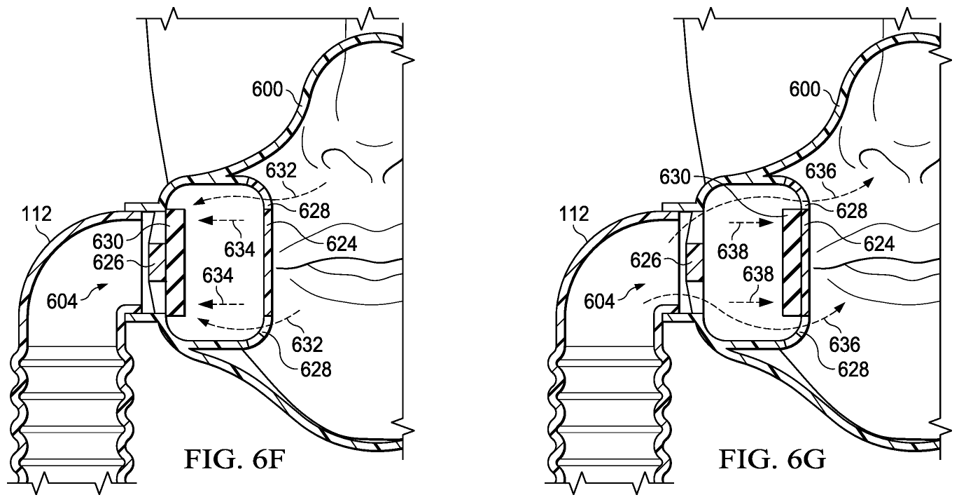

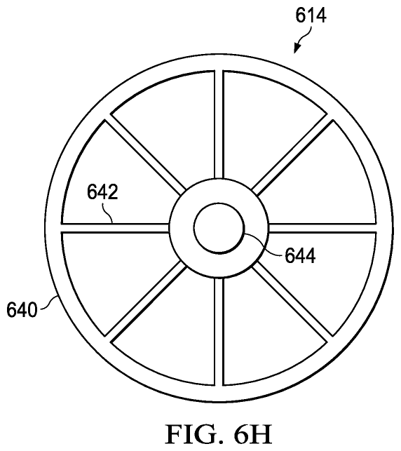

FIG. 6A is a profile view of the facemask 114 of the mask device 100 in various examples. The facemask 114 includes a mask body 600, a port 604, a valve cap 606, orifices 608, and a fastening member (e.g., an elastic fastening member) 610. The flexible hose 112 may be coupled to the mask body 600 by inserting the flexible hose 112 into the port 604. Although not expressly shown, the port 604 may include a shoulder. Further, as described below, the port 604 may include an air valve that is configured to open responsive to the wearer of the facemask 114 inhaling and to close responsive to the wearer of the facemask 114 exhaling. In this way, unsanitary air exhaled by the wearer is not provided to the flexible hose 112 or the sanitizing member 102. As described below, another air valve includes the valve cap 606 and components behind the valve cap 606, and this air valve (e.g., an exhaust valve) may be configured to open responsive to the wearer of the facemask 114 exhaling and to close responsive to the wearer of the facemask 114 inhaling. In this way, the wearer of the facemask 114 does not inhale unsanitary air. Airflow through the air valve of the valve cap 606 may pass through the orifices 608 when the valve cap 606 is closed.

FIG. 6B is a profile view of the facemask 114 of the mask device 100 in various examples. Specifically, FIG. 6B provides a view behind the valve cap 606 when the valve cap 606 is opened, or lifted. The valve cap 606 includes the orifices 608, which may be arranged along a circular path having a circumference at least as large as that of an air blocking member 612. The air blocking member 612 is positioned behind the valve cap 606 when the valve cap 606 is in the closed position. FIG. 6C is a profile view of the facemask 114 of the mask device 100 in various examples. In FIG. 6C, the air blocking member 612 has been removed to reveal a valve frame 614 behind the air blocking member 612 and the valve cap 606.

In examples, the valve cap 606 is composed of plastic (e.g., polyvinyl chloride (PVC)). In examples, the air blocking member 612 is composed of a flexible, non-porous material. In examples, the air blocking member 612 is composed of rubber. In examples, the valve frame 614 is composed of PVC. Other materials may be useful in the valve cap 606, the air blocking member 612, and/or the valve frame 614 to open and close responsive to the wearer's respiration patterns.

FIGS. 6D-6H are profile and profile cross-sectional views of air valves in the mask device 100 in various examples. Specifically, FIGS. 6D, 6E and 6H show the air valve formed by the valve cap 606, the air blocking member 612, the valve frame 614, and the mask body 600. FIGS. 6F, 6G and 6H show the air valve in the port 604.

The air valve of FIG. 6D includes the mask body 600, the valve cap 606, the orifices 608, the air blocking member 612, and the valve frame 614. The air blocking member 612 is mobile and may move between the valve cap 606 and the valve frame 614 responsive to the wearer's respiration patterns. Air may flow through the air valve via the orifices 608 and the valve frame 614. As FIG. 6D shows, responsive to the wearer of the facemask 114 inhaling (numeral 616), the air blocking member 612 moves toward the wearer and abuts the valve frame 614. Numeral 618 indicates this movement. The air blocking member 612 seals the valve frame 614, precluding air from flowing through the orifices 608 and through the valve frame 614. In this way, the wearer is precluded from breathing unsanitary air. FIG. 6E shows the same configuration as FIG. 6D, except that the wearer is exhaling (numeral 620), which pushes the air blocking member 612 toward the valve cap 606. Numeral 622 indicates this movement. Thus, air flows from the wearer, through the valve frame 614, through the orifices 608, and into the environment. The proportions of the air valve as shown in FIGS. 6D and 6E are distorted to more clearly show and describe the operation of the air valve.

The air valve in port 604 has a similar principle of operation as the air valve described above, but in the opposite direction. FIG. 6F shows a valve cap 624, valve frame 626, orifices 628 in the valve cap 624, and an air blocking member 630. Responsive to the wearer exhaling (numeral 632), the air blocking member 630 is pushed to the valve frame 626, thereby sealing off the valve frame 626 and preventing exhaled air from entering the flexible hose 112. FIG. 6G shows the same configuration as FIG. 6F, except that the wearer is inhaling (numeral 636), which pulls the air blocking member 630 to the valve cap 624. Numeral 638 indicates this movement. Thus, air is permitted to flow from the flexible hose 112, through the valve frame 626, through the orifices 628, and to the wearer.

FIG. 6H is a profile view of the valve frame 614, which may be similar or identical to the valve frame 626. In some examples, the valve frame 614 includes a circular member 640, radial members 642, and a center member 644. In examples, the center member 644 may include a protrusion that is adapted to be coupled to an orifice (not expressly shown) in the air blocking member 612 to promote alignment between the air blocking member 612 and the valve frame 614.

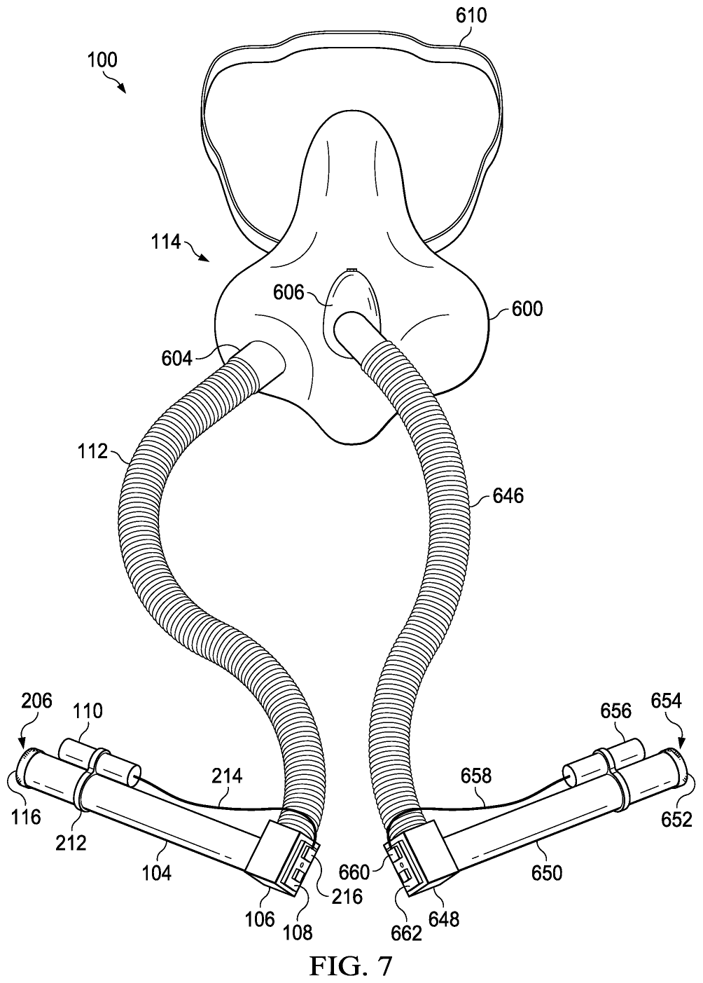

In some examples described above, exhaled air is provided into the environment of the wearer. In some cases, it may be useful to avoid expelling potentially contaminated air into the environment. In such cases, it may be useful to sanitize air being inhaled by the wearer and to sanitize air being exhaled by the wearer. FIG. 7 is a perspective view of a mask device 100. The example shown in FIG. 7 is identical to the example shown in FIG. 1 and described above, except that the example of FIG. 7 includes sanitation features coupled to the valve cap 606 to sanitize exhaled air. Specifically, a flexible hose 646 is coupled to the valve cap 606 and covers the orifices 608 (FIG. 6A-6E). The flexible hose 646 is coupled to a hollow tube end cap 648, which is similar to the hollow tube end cap 106 described above. The hollow tube end cap 648 is coupled to a hollow tube 650, which is similar to the hollow tube 104 described above. The hollow tube 650 is coupled to an optional UV blocking filter 652, which is similar to the UV blocking filter 116 described above. The hollow tube 650 includes an orifice at a distal end 654, which is similar to the orifice at the distal end 206. A battery 656 is coupled to a battery wire 658, which are similar to the battery 110 and the battery wire 214, respectively. The battery wire 658 is coupled to a connector 660, which is similar to the connector 216 described above. The connector 216 is coupled to a UV-LED assembly 662, which is similar to the UV-LED assembly 108 described above.

In operation, responsive to the wearer inhaling, the air valve at port 604 opens, and the air valve at valve cap 606 closes. Accordingly, unsanitary air enters the orifice at distal end 206 and flows through the hollow tube 104. The UV-C light emitted by the UV-LED on the UV-LED assembly 108 sanitizes the air as the air flows up the hollow tube 104. Sanitized air enters the flexible hose 112 and is provided to the wearer through the port 604. Responsive to the wearer exhaling, the air valve at the port 604 closes, and the air valve at valve cap 606 opens. Accordingly, unsanitary, exhaled air flows through the flexible hose 646 to the hollow tube 650. The UV-C light emitted by the UV-LED on the UV-LED assembly 662 sanitizes the exhaled air as the air travels along the length of the hollow tube 650, toward the orifice at distal end 654. The orifice at distal end 654 emits sanitized air.

As described above, the hollow tube 104 and the UV-LED 404 (and the hollow tube 650 with its UV-LED) may have specific parameters, such as UV-LED wattage and dimensions of the hollow tube 104, that enable sanitization of air. For example, illuminating 1 meter of unsanitary air with a 100 milli Watt (mW) UV-C light for 60 seconds (which is equivalent to 6 Watts*seconds per meter) results in a 90% reduction in severe acute respiratory syndrome coronavirus 2 (SARS-CoV-2, also known as COVID-19) pathogens. In this example, the 1 m² of unsanitary air may have a thickness equivalent to the largest dimension of a single pathogen. A greater exposure (e.g., with a higher mW UV-LED) or a longer exposure time results in more than a 90% reduction in pathogen load, and a lesser exposure results in less than a 90% reduction in pathogen load. The exposure (e.g., UV power/area, such as mW/cm² or W/m²) is determined by the UV power and inner diameter of the hollow tube 104. The total UV dose is determined by the exposure and length of the hollow tube 104 to produce a specific reduction in pathogen load. The total UV dose to reduce pathogen load may vary from pathogen to pathogen. Generally, for a given UV-LED power, airflow (volume per unit time), and UV exposure requirement (e.g., depending on the pathogen), a longer tube increases exposure time, increases UV dosage, and increases efficacy in pathogen elimination. A greater UV-C exposure reduces UV power requirement to eliminate a specific pathogen. Increasing UV-C exposure (e.g., by adding UV-LEDS and/or by increasing UV-LED power) increases battery consumption but decreases the length of the hollow tube 104 used to adequately reduce pathogen load. The diameter of the hollow tube 104 may be determined based on multiple factors, including portability (with a narrower hollow tube 104 being more portable) and air flow (with a narrower hollow tube 104 restricting air flow).

The term “couple” is used throughout the specification to include both direct and indirect connections between components. Thus, for example, a component A that has a direct connection to component B is coupled to component B. In another example, when a component A has an indirect connection to component B via component C, component A is coupled to component B.

A device that is “configured to” perform a task or function may be configured (e.g., programmed and/or hardwired) at a time of manufacturing by a manufacturer to perform the function and/or may be configurable (or re-configurable) by a user after manufacturing to perform the function and/or other additional or alternative functions. The configuring may be through firmware and/or software programming of the device, through a construction and/or layout of hardware components and interconnections of the device, or a combination thereof.

A circuit or device that is described herein as including certain components may instead be adapted to be coupled to those components to form the described circuitry or device. For example, a structure described as including one or more semiconductor elements (such as transistors), one or more passive elements (such as resistors, capacitors, and/or inductors), and/or one or more sources (such as voltage and/or current sources) may instead include only the semiconductor elements within a single physical device (e.g., a semiconductor die and/or integrated circuit (IC) package) and may be adapted to be coupled to at least some of the passive elements and/or the sources to form the described structure either at a time of manufacture or after a time of manufacture, for example, by an end-user and/or a third-party.

Unless otherwise stated, “about,” “approximately,” or “substantially” preceding a value means+/−10 percent of the stated value. Modifications are possible in the described examples, and other examples are possible within the scope of the claims.

What is claimed is:

1. A device, comprising:
a sanitizing member including:
a hollow tube having a reflective inner surface and first and second ends; and
an ultraviolet (UV) light-emitting diode (UV-LED) at the first end, the second end having an orifice exposing the reflective inner surface to an environment of the device; and
a facemask having an air valve coupled to the sanitizing member.

2. The device of claim 1, further comprising a flexible hose coupled to the sanitizing member and to the air valve.

3. The device of claim 1, wherein the reflective inner surface includes aluminum.

4. The device of claim 1, wherein the UV-LED is configured to emit UV-C light.

5. The device of claim 1, further comprising a UV-blocking filter at the second end.

6. The device of claim 1, wherein the orifice is a first orifice, and wherein the sanitizing member includes a second orifice at the first end, the second orifice having a smaller diameter than the hollow tube, the UV-LED in the second orifice.

7. The device of claim 6, wherein the hollow tube includes a third orifice between the first and second orifices, the third orifice configured to enable airflow between the hollow tube and a flexible hose coupled to the third orifice.

8. The device of claim 1, wherein the sanitizing member is a first sanitizing member, the hollow tube is a first hollow tube, the UV-LED is a first UV-LED, and the air valve is a first air valve, the device further comprising:
a second sanitizing member including a second hollow tube and a second UV-LED, the second hollow tube coupled to a second air valve of the facemask, the second UV-LED at a first end of the second hollow tube, the second UV-LED configured to sanitize air in the second hollow tube.

9. The device of claim 8, wherein the first UV-LED is configured to sanitize air drawn from the environment in the first hollow tube, the facemask is configured to provide the sanitized air to a wearer of the facemask, and the second UV-LED is configured to sanitize air exhaled by the wearer.

10. A device, comprising:
a sanitizing member having a hollow tube and an ultraviolet light-emitting diode (UV-LED), the UV-LED configured to illuminate an interior of the hollow tube to sanitize air drawn from an environment of the device as the air flows through the hollow tube; and
a facemask coupled to the sanitizing member, the facemask configured to provide the sanitized air to a wearer of the facemask responsive to the wearer inhaling, the facemask configured to expel air to the environment responsive to the wearer exhaling.

11. The device of claim 10, wherein the interior of the hollow tube includes a reflective surface including aluminum.

12. The device of claim 10, wherein the facemask includes an air valve configured to open responsive to the wearer inhaling, and wherein the device includes a flexible hose coupled to the air valve and coupled to an orifice of the hollow tube.

13. The device of claim 12, wherein the air valve is a first valve, the sanitizing member is a first sanitizing member, the hollow tube is a first hollow tube, the UV-LED is a first UV-LED, and the flexible hose is a first flexible hose, and wherein the device further comprises a second air valve configured to open responsive to the wearer exhaling, the second air valve coupled to a second flexible hose, the second flexible hose coupled to a second sanitizing member having a second hollow tube and a second UV-LED, the second UV-LED configured to illuminate an interior of the second hollow tube to sanitize air exhaled by the wearer.

14. The device of claim 10, further comprising a UV-blocking filter at an end of the hollow tube.

15. The device of claim 10, wherein the UV-LED is configured to emit UV-C light into the hollow tube with an exposure of at least 6 Watts*seconds per meter².

16. A device, comprising:
a sanitizing member including:
a hollow tube having a reflective inner surface; and
an ultraviolet light emitting diode (UV-LED) at a first end of the hollow tube, the UV-LED configured to direct ultraviolet light toward a second end of the hollow tube to sanitize air in the hollow tube, the reflective inner surface configured to reflect the ultraviolet light; and
a facemask coupled to the sanitizing member, the facemask having an air valve configured to provide the sanitized air from the sanitizing member to a wearer of the facemask.

17. The device of claim 16, wherein the reflective inner surface includes aluminum.

18. The device of claim 16, wherein the air valve is a first air valve, and wherein the facemask includes a second air valve that is configured to expel exhaled air to an environment of the device.

19. The device of claim 16, wherein the air valve is a first air valve and the sanitizing member is a first sanitizing member, and wherein the facemask includes a second air valve that is coupled to a second sanitizing member that is configured to sanitize the exhaled air using UV light.

20. The device of claim 16, wherein the UV-LED is configured to emit UV-C light.
